(19) United States
(12) Patent Application Publication
(10) Pub. No.: US 2011/0160601 A1
(43) Pub. Date: Jun. 30, 2011

(54) Wire Free Self-Contained Single or Multi-Lead Ambulatory ECG Recording and Analyzing Device, System and Method Thereof

(76) Inventors: Yang Wang, Monmouth Junction, NJ (US); Wenyu Wang, Monmouth Junction, NJ (US)

(21) Appl. No.: 12/650,528

(22) Filed: Dec. 30, 2009

(51) Int. Cl. A61B 5/0402

(52) U.S. Cl. 600/509

(57) ABSTRACT

A device, method and system are provided to continuously store and analyze single or multi-lead ambulatory ECG signals. Each ECG device is self-contained, wire-free, reusable and capable of non-invasive continuous recording, analyzing and indexing of ECG data independently. A pair of snap connectors on the device body attaches the device to standard ECG electrodes to minimize noise caused by the wires. Each device contains a real-time clock to link the ECG data and a detected cardiac event with actual date and time. The device contains a USB port to upload the data to a host system. Multiple such devices are wearable by a patient to form a multi-lead ECG system. There is provided a method to continuously record and analyze ECG data using a self-contained, wire-free ECG device or multiple such devices; and further a method to synchronize the ECG data in multiple devices to realize multi-lead ECG recording.

### BACKGROUND OF THE INVENTION

1. Field of Invention

The invention generally relates to non-invasive cardiac medical devices, in particular, a wearable, self-contained, reusable and wire-free continuous electrocardiogram (ECG) recording and diagnosis device, system and method thereof. There is provided a unique method to use multiple such devices to achieve multi-lead ECG recording and analyzing for a long period of time.

2. Background

Currently, the problem exists as to how to provide a reliable, wire-free, and smart multi-lead ambulatory ECG recording and analyzing device. All present multi-lead ambulatory ECG recording devices have long discrete electrode wires that contribute noises and artifacts due to the wire movements. Such devices are also inconvenient to a patient's daily activities. There is no true multi-lead wire-free continuous ECG recording device available.

Current continuous ECG recording devices are simply recording the signal without analysis in order to keep the completely original data. The data is thereafter processed. However, such a method of subsequent ECG data processing adds significant work and cost.

Two major types of ambulatory ECG systems for long term monitoring and recording include continuous recorders and event monitors. The continuous recorder, the traditional Holter, typically records 24 to 48 hours of ECG signal continuously. The data is analyzed after recordings for detecting arrhythmias and investigating symptoms. Another type of long term monitoring device is an event ECG monitoring system. Unlike Holter, event monitors are intermittent recorders for investigating events that occur infrequently. An event monitor can be triggered either automatically or manually. It usually has a limited loop memory to record brief and intermittent signals over a long period time for analysis.

Currently, both types of devices are portable and capable of being carried by patients. Multiple wires are connected between electrodes and the portable devices. However, as mentioned, the long discrete electrode wires contribute noise and artifacts due to the wire movements. They also interrupt a patient's daily activities since the long term ambulatory monitoring is required during a long period of time.

Recently, there have been some attempted various self-contained miniature ECG devices which integrate electrodes, a controller, memory and battery all into one small substrate. Patients can wear such a miniature device for a long period of time. However, such devices have the following limitations and disadvantages:
(1) Not Truly Ambulatory: Most of such miniature ECG devices are limited to essentially being monitoring devices which have built-in wireless transmitters. They simply relay the real-time ECG signals to a monitoring center in real-time. The patients must therefore stay within the wireless transmitting range.
(2) Lacks Intelligence: Some of such miniature ECG devices are simply signal recorders that record all data. They will generate 10 Mbyte-100 Mbyte ECG data without any analysis. Long-term continuous data is subsequently analyzed after the recording by such devices which will take hours or days depending on the signal quality and sophistication of the computer analysis system.
(3) Lacks Reliability: All prior disclosed wire free miniature ECG devices are fundamentally a single lead ECG system due to the small size of the substrate. Such devices do not provide for multi-lead monitoring and recording which is necessary for accurate diagnosis of some arrhythmia. There are immediate advantages to multi-lead monitoring for the detection and localization of acute ischemia in patients with coronary artery disease. Multi-lead monitoring also increases the reliability of basic QRS detection. In another aspect, the redundancy of a signal with multi-lead recording provides alternate information when there is an overwhelming noise in one lead and is therefore a desirable function not available with current devices.
(4) Lacking Comfort and Versatility: All prior miniature devices have built-in electrodes and adhesives. There is no single adhesive that can be suitable for all kinds of skin types over a long period time. Such prior designs have the limitation of incompatibility with and inability to be widely used by different patients. In addition, for hygienic reasons, such built-in electrode devices are disposed of and cannot be reused.

Accordingly, there exists a need for a single and multi-lead, wire-free ECG recording and analyzing device designed for widespread usage by patients with different skin types and sensitivities, which can be re-attachable to a patient after use. Further, a need exists for integrating sophisticated diagnosis algorithms from a system to an embedded controller in a self-contained device which can analyze the ECG data in real-time while recording and preserving the original data for completeness thereby saving on time and cost for post recording analysis.

### SUMMARY OF THE INVENTION

A device, system and method are provided to continuously store and analyze single or multi-lead ambulatory ECG signals according to embodiments of the invention. Each ECG device is capable of continuously recording, analyzing and indexing the ECG data independently. A pair of snap connectors attaches the device to engage standard ECG electrodes placed on the patient, to minimize noise caused by the wires. Each device contains a real-time clock to link the ECG data and detected cardiac event with actual date and time. The device contains a USB port to upload the data to a host system after the monitoring. Multiple such devices can be worn by a patient to form a multi-lead ECG system. An embodiment of the present invention provides different methods to synchronize the ECG data in multiple devices to realize a multi-lead ECG recording.

The device according to an embodiment of the present invention is a small wearable electrocardiogram (ECG) recording device which can be attached to a patient like a bandage strip. The self contained, wire-free, ambulatory ECG device provides for non-invasive continuous recording and analysis of ECG signals, the device comprises: a device body having a flexible substrate, the substrate having embedded electronic components including a microcontroller, a memory embedded with a diagnosis software, a real time clock, a functional switch, electronic circuitry, a plurality of conductors, a battery, and an interface for data communication; as well as two or more snap connectors integrated onto the substrate for engaging with two or more ECG electrodes separate from the device, the electrodes having a means to adhere to the skin of the body. The device body is detachable from the electrodes via the snap connectors and reusable with two or more new electrodes for a subsequent recording and analysis. The self-contained device is capable of continuously recording and analyzing the cardiac data (single or multi-lead ambulatory ECG signals). Because the device is wire free and self-contained, a patient is not constrained to any hospital bed or to any larger machine.

According to another embodiment, there is a system of ECG monitoring and recording disclosed in which the system comprises: a self-contained, wire free, ambulatory single lead ECG device which continuously records and analyzes ECG data, the device comprising electronic components operatively coupled and embedded within a flexible substrate of the device, the components including an embedded microcontroller, circuitry and conductors, a real time clock, a data communication interface, memory and software, and at least two electrode snap connectors disposed on the substrate of the device; a pair of electrodes for engaging with the snap connectors of the device, each of the pair of electrodes having a means for adhering to a patient's skin. The system further comprising a computer host system having one or more processors for executing commands that direct operations of the computer system and software executing within the one or more processors that directs the one or more processors to: obtain recorded analyzed ECG data from the device through the data communications interface; calculate a relative time offset of the real time clock of the device; update all real time analyzed ECG data of the device with the calculated time offset; and display the analyzed ECG data from the device.

There are a number of features in which make this invention unique from other prior art ECG devices. A first object of the present invention is to provide a wire-free ECG recording and analyzing device. In the true sense of a wire free device, the present invention ECG device is fully self-contained. There are no moving wires. between electrodes and the device making the device comfortable and convenient to use, especially over a long period of time. The device according to an embodiment of the present invention is capable of much more than traditional “wireless” ECG. The traditional wireless ECG transmits real-time data from a device on the patient to a base unit via radio waves. The device according to the present invention, on the other hand, does not transmit any real-time data to a base unit. The data is stored and analyzed within the device. No peripheral machine is needed to analyze the data. In an embodiment, the device includes a USB port which allows for easy upload of the analyzed data to a computer. Since the data is already analyzed within the self-contained device, no additional time is needed to process the data.

According to another aspect of the invention, because there is no real-time data being transmitted to a peripheral machine, base unit, or computer, the patient is not tethered to any object. The device renders the patient ambulatory. Because a traditional wireless ECG machine is constantly transmitting real-time data to a base unit, the patient must stay within a certain range to ensure that the RF (radio frequency) signal reaches the base unit. Although the patient is not restricted to a hospital bed, the range of a traditional wireless ECG is still relatively limited. In contrast, the device according to an embodiment of the present invention will record data without restricting the patient to any particular area. The patient could arguably go about his daily routine. This can be very significant in light of the fact that the tests require the patient to be monitored for over a twenty-four (24) hour period. Even with a traditional wireless ECG device, the patient would still be bound to the limits of the hospital, or confined to the patient's room. The self-contained device according to an embodiment of the present invention allows the patient to have the freedom to go about his own business for the 24 hour or a much longer (7 day) period. This encourages patients to have such tests done on a more regular basis.

According to another aspect of the present invention, the device also digitizes the ECG signal. Continuous recording of the ECG signal occurs without interruptions providing a more accurate account of the patient's condition.

In another aspect of the present invention, the device, not only records the data, but it analyzes the data to determine if there are any abnormal heart patterns with the patient. Typically, in a traditional wireless ECG device, the analysis is done in the base unit which is separate from the patient. In a traditional ambulatory ECG device like a Holter, the analysis is done either manually or automatically by an ECG analyzer after the recording. Depending on the length of a recording period, signal quality and analyzing methods, analyzing such data could take upwards of days to process and contributes to additional cost. However, since the data is already analyzed in the device of the present invention during the recording, no additional time is needed to process the data, saving costs. While recording data, the device is able to use an algorithm which is able to determine if there are any abnormalities of the heart's rhythm. The device is able to classify and diagnose heart arrhythmias and other irregularities such as asystole, paroxysmal bradycardia, and atrial fibrillation, etc.

A further object of the present invention is to provide a device that is reusable. Some previously attempted self-contained wearable ambulatory ECG devices have built-in electrodes as part of the instrument. Therefore, the entire instrument must be disposed of after use since the electrodes cannot be used again. (Not only has the adhesive been used, but it would also be unsanitary to reuse the electrodes.) According to an embodiment of the present invention, the electrodes of the device are provided separately from the device body itself which contains the circuitry. The separate electrodes are attachable to the patient by an adhesive, and on the back of the device body are electrode snap connectors. Once the discrete electrodes have been placed onto the patient, the device can be snapped into position with the electrodes. After the data has been gathered about the patient's heart, the device can be detached from the electrodes. The electrodes can be pealed from the patient's body and thrown away. The device could be reused for a subsequent patient with a new set of electrodes. Such a configuration allows for different electrodes as needed to meet the needs of the patient and for different adhesive options on the back of such electrodes for varying patient sensitivity, thus allowing for more versatility since the entire device body need not be configured to fit a skin type.

In another aspect, there is provided multiple devices of the present invention to be used at the same time on the same patient to gather multiple ECG data from different torso locations. The prior type of wearable ambulatory ECG device has created a larger substrate that contains multiple (more than two) electrodes. Therefore, the prior device needs to be expanded in the sense of physical dimension, number of the A/D channels, and memory size, etc. to handle more electrodes. Due to the limitations of the substrate, it is not possible to locate all the necessary electrodes at the ideal locations at the same time in clinical applications. In contrast, in order to achieve multi-lead ambulatory ECG recording, according to an embodiment of the present invention, the substrate does not need to be enlarged and the device does not need to be changed. The patient simply needs to add more devices onto his/her body. The devices of the present invention are identical from a hardware and firmware point of view. Physicians have the flexibility to put any number of the devices on any available location according to clinical requirements. On the other hand, manufacturers and the health care system could benefit from higher volumes of a fewer number of different products.

The devices according to an embodiment of the present invention, each have an internal real-time clock which will record the actual time along with ECG data. With the internal clock, if the patient recorded the time of day in which a particular heart concern was observed, the patient could tell his/her physician who could in turn use that time stamp as a point of reference to review the data. Furthermore, using the internal clock and one of the synchronization approaches of the present invention, the ECG data from different devices in a multiple-lead ECG system can be synchronized. With adequate resolution of the internal real-time clock, the time registered ECG data and analyzed results from different devices can be synchronized accurately. Multi-lead ambulatory ECG recording greatly assists a physician with making a more accurate diagnosis, or provides additional insurance in the event that one device might fail or give false data or distorted data.

In a further object of the present invention, by using multiple identical devices for multi-lead purposes, it allows for ease of placement of multiple said devices on the patient's body. According to an embodiment of the present invention, there are two electrodes on a single device and each separate device can be synchronized, therefore the placement of one device is not dependent on the placement of other devices, thus providing significant flexibility.

In accordance with an embodiment, there is disclosed a multi-lead ECG monitoring and recording system comprising: a plurality of self-contained, wire free, ambulatory single lead ECG devices as described in the present invention, each device continuously records and analyzes ECG data, each device having at least two electrode snap connectors disposed on the substrate of the device; a pair of electrodes for engaging with the snap connectors of the device, each of the pair of electrodes are adhered to a patient's skin; and a computer host system having one or more processors for executing commands that direct operations of the computer system and software executing within the one or more processors. The software directs the one or more processors to: obtain recorded analyzed ECG data from each device through the data communications interface; calculate a relative time offset amongst each real time clock of each device; update all real time analyzed ECG data of the devices with the calculated time offset; correlate the analyzed ECG data from each device into a multi-lead ECG data array; combine the analyzed ECG data of each of the devices and display the analyzed ECG data from the devices.

Accordingly, there is a computer implemented method disclosed according to an embodiment of the present invention, for continuous ambulatory ECG recording and analysis, using a wire free, self contained ambulatory ECG device having embedded electronic components operatively coupled, including a microcontroller, memory with an embedded diagnosis algorithm, a real time clock, a functional switch, a battery, electronic circuitry, a plurality of conductors, and an interface for data communication. The method comprises the steps of: adhering a pair of electrodes onto a patient's skin; engaging the device with the pair of electrodes through a set of electrode snap connectors integrated onto a bottom side of a flexible substrate of the device; acquiring ECG signals through the electrodes and recording the signals as raw ECG data temporarily in a memory; electronically analyzing the raw ECG data in real-time resulting in analyzed ECG data, through the microcontroller; electronically registering the analyzed ECG data with a time stamp with the embedded real time clock, through the microcontroller; electronically indexing the raw ECG data with the analyzed ECG data and the real time clock, through the microcontroller; and electronically storing the indexed and analyzed ECG data into the non-volatile memory.

According to embodiments of the present invention, the device is designed in such a way that no other extra device is required for operating and acquiring ECG data and a patient could purchase the device over-the-counter. It is a very low cost device and can be used to collect data and operate at home without training (similar to today's blood pressure monitor). The ECG data and automatic analysis results (the “analysis results” also referred to as “analyzed ECG data”) are downloaded from the device to a computer or a patient could hand over the device to a physician for a final diagnosis.

These and other embodiments of the present invention are further made apparent, in the remainder of the present document, to those of ordinary skill in the art.

### BRIEF DESCRIPTION OF THE DRAWINGS

In order to more fully describe embodiments of the present invention, reference is made to the accompanying drawings. These drawings are not to be considered limitations in the scope of the invention, but are merely illustrative.

FIG. 1 illustrates a plurality of devices set on the human body, according to embodiment of the present invention.

FIG. 2A illustrates a top view of the device showing on-board electronic components, according to an embodiment of the present invention.

FIG. 2B illustrates a side view of the device as shown in FIG. 2A and corresponding electrodes for connection, according to an embodiment of the present invention.

FIG. 3 illustrates a perspective view of the device, according to an embodiment of the present invention.

FIG. 4A illustrates a top view of the device showing on-board electronic components, according to an embodiment of the present invention.

FIG. 4B illustrates a side view of the device as shown in FIG. 4A according to an embodiment of the present invention.

FIG. 4C illustrates a side view of the device as shown in FIG. 4A according to another embodiment of the present invention.

FIG. 5 illustrates a block diagram of the electronic components of the circuitry of the device, according to an embodiment of the present invention.

FIG. 6 illustrates the system in connection with the device for retrieval of analyzed data and a synchronization of a plurality of devices, according to an embodiment of the present invention.

FIG. 7 illustrates a synchronization of a plurality of devices, according to an embodiment of the present invention.

### DETAILED DESCRIPTION OF SPECIFIC EMBODIMENTS

The description above and below and the drawings of the present invention focus on one or more currently preferred embodiments of the present invention and also describe some exemplary optional features and/or alternative embodiments. The description and drawings are for the purpose of illustration and not limitation. Those of ordinary skill in the art would recognize variations, modifications, and alternatives. Such variations, modifications, and alternatives are also within the scope of the present invention.

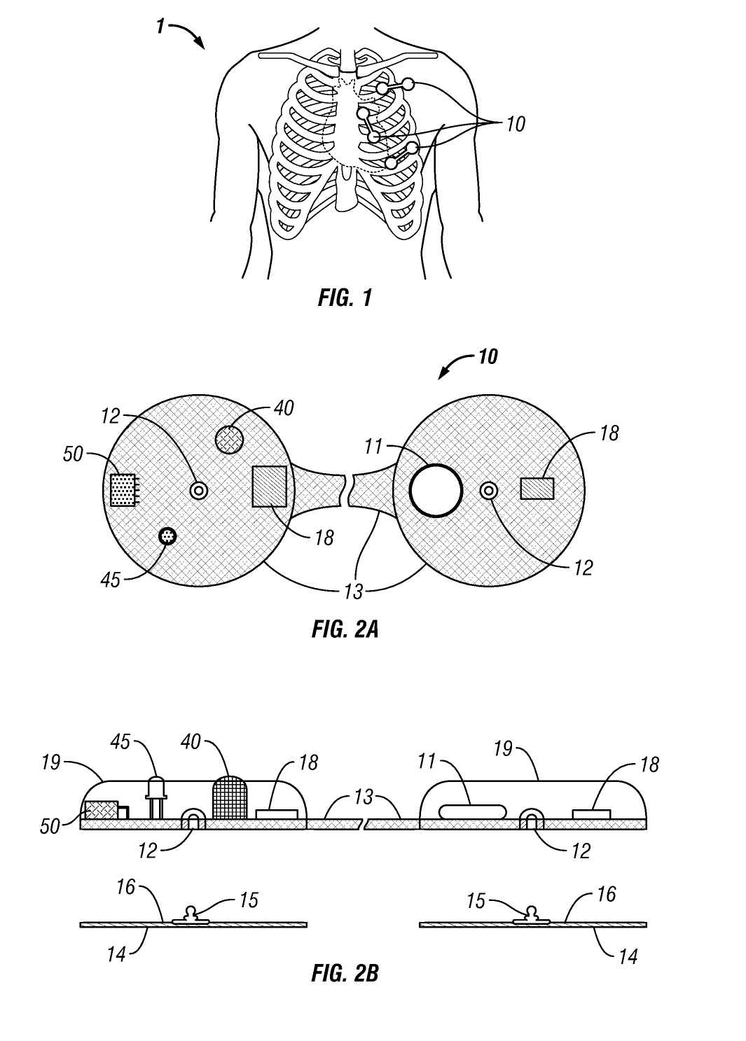

As shown in FIG. 1, a plurality of devices 10, according to an embodiment of the present invention, may be placed on the body 1 and shows the self-contained devices capable of multi-lead ability without constriction or dependency on placement of any other device having a pair of electrodes. The number and location of devices 10 placed upon the body 1 is not limited to any particular depiction shown in the figures.

A single device 10 according to an embodiment of the present invention is shown in FIGS. 2A-2B as an integrated wearable self-contained ambulatory ECG continuous recording and analyzing device built on a flexible substrate 13. The device contains at least two snap connectors 12 (or pinch connectors) integrated and located on a surface of the substrate 13 for engaging with separate electrodes 16. The user can select different electrodes 16 depending on the length of the recording and skin sensitivity. An advantage of the present invention is that the device can use industrial standard electrodes and take advantage of mature electrode design and materials. Unlike the built-in electrode monitoring devices from prior solutions, a user of the device of the present invention can select from various electrodes depending on one's specific needs. For example, it makes little sense to use a long-term electrode for a test that only takes 15 minutes. Further, even during short tests, some patients are resting whereas others are exercising and sweating from exertion. Each type of application demands different properties from the electrodes.

Since the electrodes are a separate component from the device, there are various commercially available electrodes that can be used. In one embodiment, to achieve good signal quality, silver chloride gel electrodes can be used as one of the ideal types of electrodes because they offer a low offset voltage and are reliable in reducing motion artifact.

The device 10 itself does not contain any conductive gel for electrode contacts or adhesive for the device 10 to attach to person's body. It is only the separate electrodes 16 which are gel electrodes, and which include a means for adhering to the skin, such as an adhesive on the backside of the electrode 16 itself for the necessary attachment to the body. The device 10 attaches to the person's torso through the snap connectors 12 and electrodes 16. The device provides flexibility for a user to change the electrodes as needed at the beginning or during the course of recording.

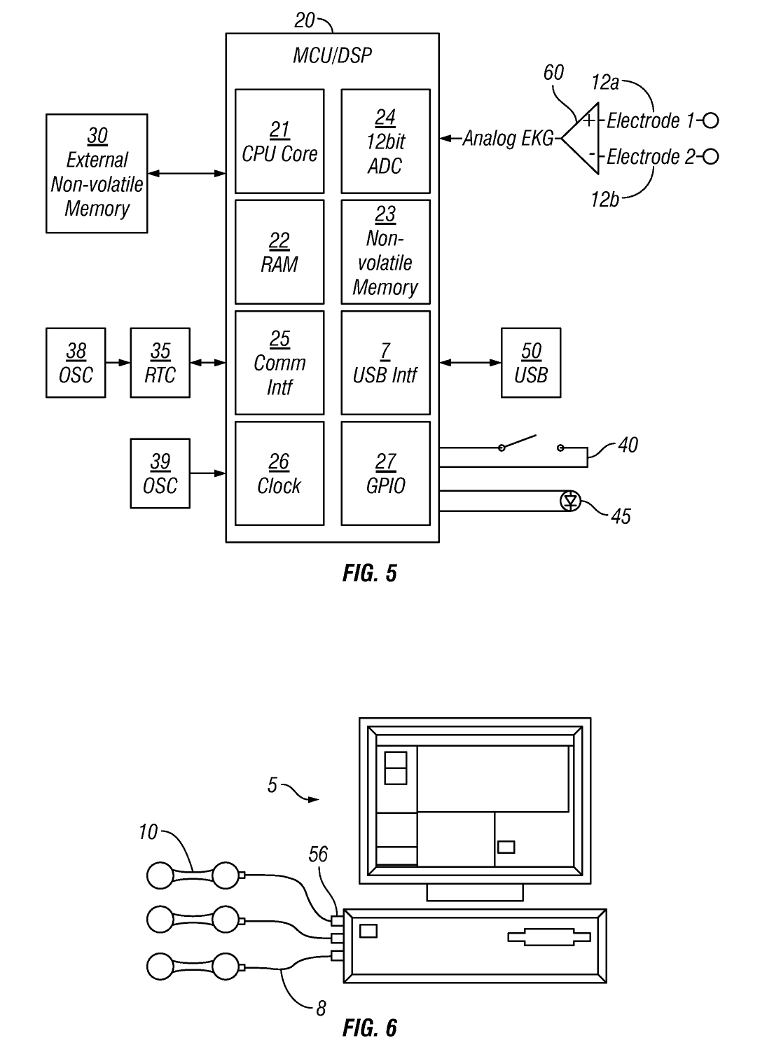

FIGS. 2A, 2B and FIG. 5 show the on-board major electronic components of the device according to an embodiment of the present invention (FIG. 5 provides a block diagram). The embedded components of the device comprise ECG front end circuitry 60, microcontroller (MCU) 20, external non-volatile memory 30, real time clock (RTC) 35, functional switch 40, LED 45 and USB interface connector 50. As shown in FIG. 2B, in a side view of the device according to an embodiment, the battery 11 is also built onto the same substrate 13. The electrical connections among the electrodes 16 and the control components are integrated inside the substrate 13 of the self contained device 10. The substrate 13 can be a flexible printed circuit board (PCB) and/or other soft material 19 such as thermoplastic elastomer or silicone which encapsulates the components and small conductors. In one aspect, the flexible substrate 13 makes the electrode placement more flexible by facilitating the fit of the device tightly around the different curves of the body.

Unlike traditional ECG recording systems with long electrode leads bridging between electrodes and its recording device, the integrated design of the embodiment of the present invention localizes the functions such as monitoring, recording and analyzing the ECG signal around the electrodes. The conductors between the electrode connectors 12 and circuitry 18 are embedded inside the flexible and/or soft substrate 13. (Circuitry 18 refers to all embedded circuitry including the MCU, memory and front end circuitry etc.) The design effectively shortens the length of the conductors and eliminates the relative movements between the wires. It also eliminates or minimizes the relative movements between lead wires and electrodes. There are no moving conductors inside the device. Therefore, motion artifacts due to movement of lead wires and electrodes themselves, which can cause large amplitude spikes on the ECG signal, various noises, wandering baselines, small complexes and fuzzy tracings can be eliminated. Some of the motion artifacts are in the same frequency ranges of valid ECG signal and are very difficult to remove by either electronic filters or algorithm. Since motion artifacts can be very prevalent in ambulatory monitoring and recording systems where some of the artifacts mix with ECG signals, it is very important and beneficial to minimize or eliminate such artifacts. The self-contained, integrated design of the embodiment of the present invention provides a superior quality of ECG signal, compared to traditional ambulatory ECG devices.

The device 10 essentially integrates and miniaturizes the ECG leads and device into a single small flexible substrate 13. In one preferred embodiment of the present invention, the device uses two ECG electrodes 16 for each device. For reliable skin contact, standard gel electrodes are recommended and a patient's skin is prepared under standard requirements. The electrodes 16 are separated in the range of about 3 to 5 inches with a minimum allowable distance of 2 to 3 inches. Since the device does not have built-in electrodes, but rather two or more snap-on interface connectors 12 to engage the separate electrodes 16, there is no adhering surface on the bottom or perimeter of the device body. The device is attached to the patient through the connectors 12 attaching to the two adhering electrodes 16 having a push button interface 15 and adhesive surface 14, as illustrated in FIG. 2B. It becomes a wearable patch connected by way of the two adhering electrodes 16. The interface approach makes the device reusable since the used electrodes may be disposed of and the device reused. The conventional wearable ECG type patches, as discussed, have built-in electrodes with a self-adhering surface. Such prior devices can only be used as a disposable device since the adhesive on the device cannot be reused.

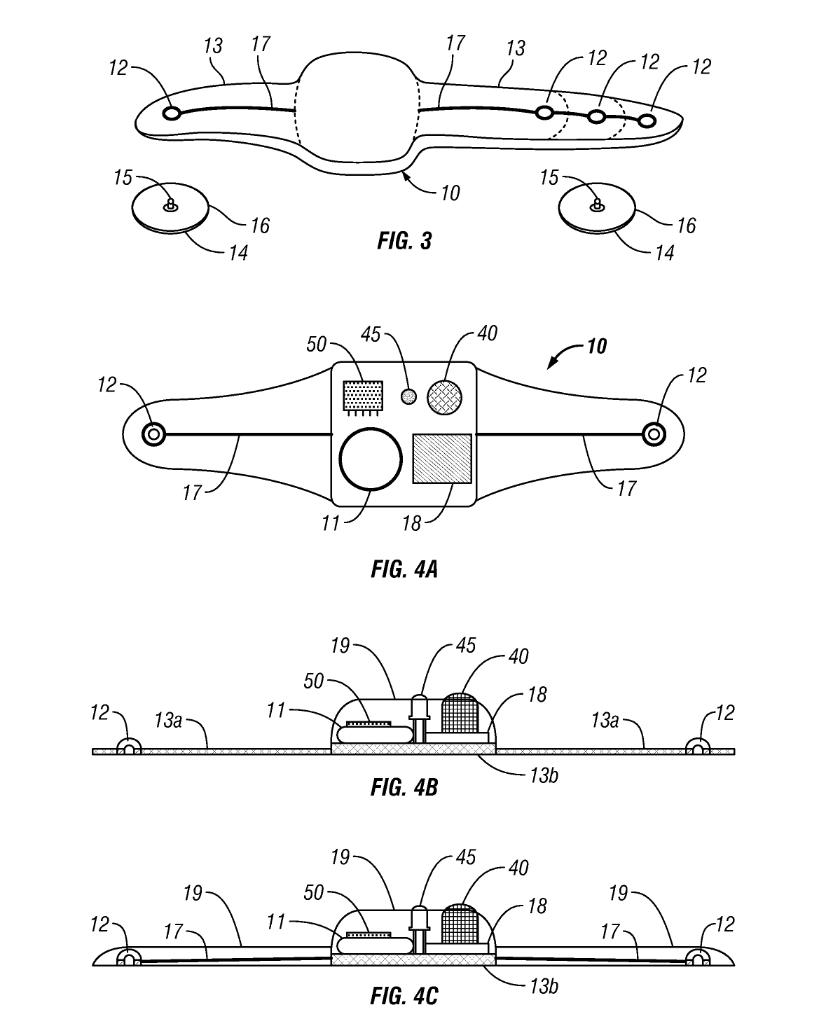

As further illustrated in FIG. 3 a perspective view of device 10 with multiple snap-on connectors 12 and two standard ECG electrodes 16, in one embodiment, the circuits 18 and battery 11 are located in the center part of the device. The components are encapsulated or potted with soft material such as silicone. The over mold material not only adds functionality like waterproofing, vibration dampening but also creates texture or a soft touching surface. At the same time, it increases the local rigidity for the electronics. Furthermore, by using different materials and combinations, the device is provided with other characteristics such as breathability or elastic features which result in further comfort for users. The snap-on connectors 12 are connected with the circuitry 18 through flexible PCB substrate 13 or conductors 17 inside over mold soft material 19. Two standard ECG electrodes 16 are shown in the drawing. Each electrode 16 contains a push button interface 15 and adhesive 14 on its backside. The device 10 attaches to both electrodes 16 and adheres to the patient's body via the electrodes 16.

Commercial ECG electrodes come in all sorts of shapes, sizes and with many different features. Most of them come with a standard push button. Patients can select different electrodes depending on the requirements needed. The approach in using standard ECG electrodes, instead of build-in electrodes, makes the device of the present invention suitable for wide range of applications.

In another embodiment of the present invention, the device body comprises one snap-on connector 12 at one end of the device and one or more connectors 12 on the other end of the device. See FIG. 3. The extra unused outside connectors can be removed or cut depending on the patient's body size. Having multiple positions where electrodes can be snapped onto results in a better fit for different body size and curves. The multiple allowable distances in the device are calibrated and the circuits can adapt the signal strength variation in those ranges. The unique interface design makes one device suitable for different physical body sizes from infant to adult.

Furthermore, since an ECG measures and displays the voltage between pairs of electrodes, and the electrical activity of a heart from different directions, also understood as vectors, the direction of the device or electrode placement is important for capturing valid and meaningful ECG signals. Accordingly, the direction of the electrodes would be fixed relative to the device of the embodiment of the present invention. It makes the placement more intuitive and easier, compared to past traditional electrode placements.

As illustrated in further detail in FIG. 4A, according to an embodiment of the device, embedded components are shown in a centralized part of the device in which the snap connectors 12 connected to the circuitry with conductors 17. In addition, the different substrate configurations are shown in FIGS. 4B and 4C. As mentioned, one approach is to use rigid-flex PCB. As shown in FIG. 4B, the device is shown with a rigid-flex PCB according to an embodiment of the present invention. The rigid-flex PCB is a binding of a flexible printed circuit board 13a and rigid printed circuit board 13b structure together in a same unit. The electronic circuitry 18 is located on the central rigid PCB 13b and snap-on connectors 12 are located on the lateral flexible PCB 13a.

Another approach is to encapsulate the connectors 12 and associated conductors 17 into a soft material 19 such as silicone instead of flexible PCB 13a, as shown in FIG. 4C. The central rigid PCB 13b can also be over mold with the soft polymeric material 19.

In both implementations, the connectors 12 and associated short 17 conductors are embedded inside a flexible material 19 (polymeric material or flexible PCB). Once the device is attached to the body, there are no relative movements among them during the monitoring period.

The device according to an embodiment of the present invention performs continuous processing, continuous monitoring, continuous analyzing and continuous recording of ECG data. Unlike any other conventional ECG devices, the device analyzes and indexes each of the data before storing them sequentially by taking advantage of the capability of the microcontroller (MCU) or digital signal processor (DSP). Traditional ECG monitoring systems store either the continuous data without analysis or records intermittent event data only (when it is triggered). One of the merits of the present invention is that indexing the ECG data greatly simplifies the time and cost for the subsequent data processing. The computing power of using an embedded MCU or DSP enables real-time ECG data analysis within the device itself besides conventional digitization and storage of data functions.

As shown in FIG. 5, a real time clock 35 is used to time index the ECG data. The real time clock (RTC) 35 used in the embodiment of the invention provides a real time stamp for each data either directly or indirectly. Whenever the user pushes the functional switch 40 to activate a continuous ECG monitoring and recording process, the first data train stored into the memory is real time clock data instead of ECG data. The unique index value differentiates RTC data from ECG data. The RTC data includes calendar year, month, date and time. The accuracy of the real time clock can be up to 1 ms. The ECG data is always sampled at a fixed rate which is normally between 5-10 ms intervals. Once the first continuous ECG data is registered with the real time clock, the time for the rest of the sequential data can be derived from the location of the memory and the first RTC value.

In the device of an embodiment of the present invention, there is provided an approach to stamp each recorded ECG data with the real time clock without using large amount of memory. The device uses an embedded real time clock to stamp the ECG data and provides a unique method to sort the data by the calendar date and time. A user can record the time when feeling symptoms during the monitoring process and inform the physician at a later time. The physician is able to retrieve the data accordingly. The microcontroller or DSP calculates the location of the data according to the first real time stamp. The ECG data corresponding to the particular date and time can then be determined and uploaded from the memory.

According to an embodiment of the present invention, a multi-lead ECG monitoring and recording system using a plurality of the devices described is provided. The plurality of devices are attached to different locations of a person's torso. The multi-lead ECG monitoring system is a wearable and wire free ambulatory ECG system. By placing the multiple devices in the different body locations, each ECG device can monitor and record the local ECG data individually and independently of each other.

The multi-lead system uses the embedded real time clock 35 within each device 10, as well as a synchronization process to synchronize the multiple devices. The plurality of independent devices and their recording signals/information are therefore accurately correlated to each other. Reliable and adequate information can therefore be obtained for a physician. The multi-lead wire free continuous ECG recording and analyzing method is an advanced approach for long term ambulatory ECG monitoring. The method does not simply add more ECG signals to a single ECG device via additional wires like a multi-lead Holter. Prior solution attempts either use multiple wires connecting between electrodes and a portable device such as a Holter or are a single lead self-contained device. Single lead ECG device are not sufficient for diagnosing certain cardiac problems and are not reliable as a sole signal source in some cases.

Furthermore, there are no physical connections or wires among the devices in the ambulatory multi-lead ECG system of the present invention. Each device is configured to detect, acquire, analyze, and record the individual ECG vector on its own independent of each other. The devices in the system can be synchronized before they are attached to a person or after they are removed from a person. The multi-lead ECG data from different devices are combined together according to the individual real time clock 35 and a reference signal or reference clock in an external host system or a computer.

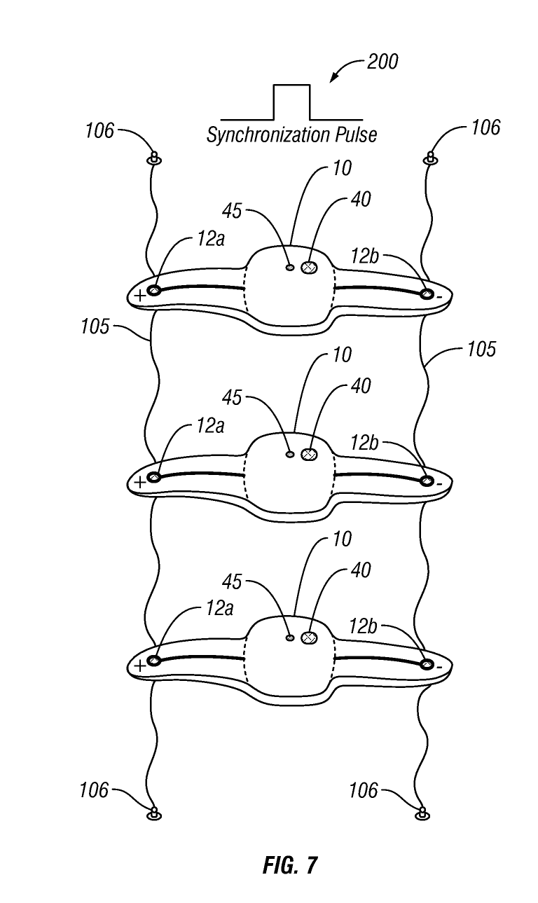

The absolute real time clock 35 of the device is not accurate enough to be used as a synchronization reference directly between different devices in the multi-lead ECG system. The embodiment of the present invention discloses one method to synchronize different devices of the present invention to a same triggering signal and compensate the error of RTC. In synchronizing all the devices in the multi-lead ECG system shown in FIG. 1, the synchronization process derives the relative offset information among the real time clocks in the different devices. A synchronization process of the multi-lead ECG system according to an embodiment is described in more detail with respect to an example in FIG. 7 below. The synchronization process can be performed before placing the devices onto the patient or after their removal from the patient.

In FIG. 7 a synchronization process of the internal clocks on the devices for a multi-lead ECG system according to an embodiment of the present invention is now described in further detail. The method of synchronization involves the following steps:
(1) Connect the positive electrode snap-on connector (12a) from different devices (10) together with a provided electrical conducting wire (105). The conducting wire (105) contains complementary pins (106) to snap fit the connector (12a) on the device (10).
(2) Connect the negative snap-on connector (12b) from different devices (10) together with a separate provided electrical conducting wire (105). The conducting wire (105) contains complementary pins (106) to snap fit the connector (12b) on the device (10).
(3) Set all the devices (10) in the synchronization mode (their default states are slave devices) using functional button (40).
(4) Change any one of the said devices (10) as a master device using the functional button (40).
(5) Send out a particular pulse or pulse train 200 from the master device using the functional switch (40). Once the master and slave devices (10) receive the expected particular pulse or pulse train 200, each device will record the time from their own embedded RTC with an RTC synchronization index. This time will be used as synchronization reference.
(6) If the device is successfully synchronized, the LED (45) will show the device (10) in synchronized mode. If all the devices (master/slave) (10) show they are in synchronized mode, the process is successful. Otherwise, the process is repeated.
(7) Reset the master device and slave devices (10) back to the active state by using the functional switch (40). The active state allows for data retrieval and recording by the device.
(8) Removing the connecting wires (105).
   The devices as shown in FIG. 7 are thereby synchronized to a same triggering signal by this method using the external apparatus.

According to another embodiment of the present invention, the synchronization is achieved without an apparatus and conducted through a computer or an external system 5. By using a time from the system 5 as a reference, all the devices in the multi-lead system can be synchronized to that time clock from the system 5. The time clock can be any accurate time for example, Network Time Protocol (NTP), which includes a 64-bit timestamp with a resolution of 200 ps or simply the computer clock. Such clocks are accurate enough to be used as the ECG synchronizing reference. The synchronization process can be done prior to the usage of the devices or immediately after usage, such as during the data retrieving stage. The device is interfaced with the system 5 via USB port 56, as shown in FIG. 6. The proprietary system software will register the device's time clock with the above accurate time reference. The offset between the two clocks are obtained. The similar offsets for other devices can be obtained in the same way.

As discussed, an embodiment of the present invention comprises a multi-lead ECG monitoring system with multiple identical devices attached to a patient. As mentioned, all the devices in the system are to be synchronized before placing them onto a body or after removing them from a body. The synchronization processes described previously has each device record a time corresponding to a same triggering pulse or pulse train or to the same accurate time reference. This information provides a relative offset amongst real time clocks in the different devices. By placing the multiple said devices in the different body locations, each ECG device can detect and record the local spatial ECG information individually. By synchronizing and analyzing the ECG data from different devices, it provides many more valuable perspectives to monitor heart activities compared to single lead ECG.

After recording, the ECG data and synchronization information are retrieved from all individual said devices to the host computer or system 5 shown in FIG. 6. Retrieval of the information from each device 10 is provided, for example, through a corresponding USB cable 8 and USB port 56 on the system 5. Since each ECG data is registered with its own real time clock (RTC) 35 and each RTC has been synchronized to a same triggering signal or a same accurate time reference, the relative time offset among said devices can be calculated. The recorded ECG data from different devices 10 that are registered with their own real time clock can be correlated and reconstructed to a multi-lead ECG data array with help of the application software at the host computer or system 5. The present invention provides a unique method to perform true wire free multi-lead ECG recording and analyzing with multiple single lead ECG devices of the present invention. Accordingly, long term multi-lead ambulatory ECG monitoring is uninterrupted and convenient.

Returning to FIG. 5, the microcontroller or DSP 20 is illustrated as a single chip having functional blocks within. The microcontroller/DSP 20 comprises internal RAM 22 and flash memory 23 operatively coupled inside the microcontroller 20 such that the RAM 22 and flash 23 are connected to the CPU core through an internal bus inside the chip. It has at least one-channel 12-bit analog-to-digital converter (ADC) 24. After the ECG signal is processed and amplified by the front end circuitry 60, a 12-bit analog-to-digital converter 24 digitizes the signal and stores them into data RAM 22 of the microcontroller or DSP 20 temporarily. An embedded cardiac diagnosis algorithm resides in the internal flash memory 23 of the microcontroller or DSP 20. Unlike any other conventional ECG devices, the embedded algorithm not only records but detects, analyzes and indexes ECG data in real-time by using the microcontroller or DSP 20. Annotation for the cardiac rhythm associated with a unique index is inserted into the data memory. The index is an identifier for the annotation result. It distinguishes the annotation data from raw ECG data. Traditionally, the ECG monitoring system stores either the continuous data without analysis or intermittent event data only. By indexing the ECG data with analysis results of the embedded algorithm, the embodiment of the present invention here not only simplifies the complexity of following data processing but preserves the original continuous data for completeness. After the ECG data has been analyzed, they are transferred into an external non-volatile memory 30 from internal RAM 22. The oscillator 38 provides a clock reference to the real time clock (RTC) 35, which communicates with peripheral communication unit 25 of MCU/DSP 20 via a serial communication interface such as I²C or SPI. The oscillator 39 serves as a clock generator to the clock interface 26 of MCU 20. The switch 40 and LED 45 are interfaced with the general purpose I/O (GPIO) 27. The external non-volatile memory 30 is connected with MCU/DSP 20 through either a parallel or serial bus. The recorded ECG data and analyzed results (the “analyzed results” referring to the “analyzed ECG data”) are stored in the above memory 30.

The device includes a standard built-in USB interface connector 50 interfaced with USB interface 7 of the MCU 20. The primary function of the interface connector 50 is to upload the analyzed ECG data from the external flash memory 30 to the host system 5 shown in FIG. 6. The selection of an industrial standard interface makes the device capable of adapting to many other existing devices in many aspects. It also can be used as a functional expansion port for the invented device. Many functions that are not essential to the primary function of the device can be added to it via USB interface connector 50 in some applications. It also makes the invented device usable as a storage media for the patient's record after finishing the monitoring and recording tasks.

The functional switch 40 is intended for execution of primary as well as expansion functions. The primary function of the switch 40 is to activate or deactivate the recording of the device. The switch 40 can also be pressed in different patterns to activate different predefined functions accordingly. The embedded microcontroller or DSP 20 is able to capture the patterns sent by the switch 40 at real time and execute the corresponding tasks. The tasks include activating the LED 45 to inform the user about the present status.

The embodiment of the invention includes feedback for the device. The feedback can be a visual indicator such as LED 45 or an audio alarm such as a micro buzzer. The feedback indicator can reflect the status of the device. For example, the LED 45 can blink once for each detected heartbeat or on other conditions. The color of LED 45 can also indicate whether the device functions correctly or not. The indicators are used as state and functional indications such that the device is in the synchronized mode, or the device is in power-on state or the device is in the recording mode, etc. The combination of colors and blinking patterns can be used for indicating different functions or status. It is important for a user to know whether the device is able to detect the cardiac rhythm or not. The embodiment of the invention also provides that the functional switch can disable such feedback indications to reduce power usage.

Although the device is capable of determining that an abnormal condition is occurring during the monitoring of a patient, the device is intended to capture and document any temporary or intermittent abnormalities such as irregular heartbeats. Therefore, the device is ideal for outpatients. Any abnormal conditions usually would not be handled by the patients themselves. Neither do many arrhythmias need immediate attention either. The real-time analysis in the device of the present invention is intended to save time and cost in post data processing.

The miniature battery 11 provides power for all the electronics in the device. The battery 11 shall be able to supply the device both in standby mode (when it is in the shelf) for at least 1 year and in active mode for at least 7 days. The device does not need battery power for data uploading. The host computer or system will power the device once it plugs in via USB interface connector 50. This makes sure the recording data will be able to upload even if the on-board battery has run out.

As described, each device 10 contains a real time clock (RTC). Its current consumption is very low for today's low power RTC chip. The current consumption of those integrated circuits can be less than 500 nA. For a one-year shelf time, the RTC alone consumes only 4.38 mAh of power. It only represents about 2% of a battery capacity with a current capacity being of 200 mAh.

During the fabrication of the device, the real time clock can be accurately set up by its manufacturer. However, the industrial level RTC normally generates a large error over time due to the variations of crystal and temperature. For example, the DS3232 from Dallas Semiconductor can achieve accuracies of better than ±1.8 min/year over an entire industrial temperature range (−40° C. to +85° C.). They are not accurate enough to be used as a synchronization reference directly for the heart beat between different devices in a multi-lead ECG system. The embodiment of the present invention provides a method to synchronize different devices of the present invention to a same triggering signal and compensate the error of RTC. The synchronization can be done prior to or immediately after usage. The triggering signal can be generated in different ways either by an internal signal source or external signal source. The triggering pattern could be a single pulse or a particular pulse train. The triggering inputs can be through electrode connectors or through other ports. An embodiment of the present invention also provides a method to synchronize different devices of the present invention to a same time reference or clock from the external system or computer.

Once all the ECG data are loaded into the system after finishing the recording, (see FIG. 6), the algorithm will calculate the relative time offsets between each device according to the synchronization information. One exemplary implementation is to take a RTC from one device as reference and change the real time information of the rest of devices according to the calculated offset. Under the new time reference, all the ECG data (consisted by many sample points) are aligned to a same reference and constructed into a multi-lead ECG data array. The data array can be displayed conventionally as an ECG graph. The external ECG system also has the functions to extract the analysis results from each individual device and combine them together. If necessary, more sophisticated ECG analysis algorithms can be used in the external system to further analyze the data from different devices.

The ECG records the electrical activity that results when the heart muscle cells in the atria and ventricles contract. The appearance of ECG depends on where on the body the measuring electrodes are placed. The standard 12-lead electrocardiogram is a typical representation of the heart's electrical activity recorded from electrodes on the body surface. It provides spatial information about the heart's electrical activity in approximately orthogonal directions: right-left, superior-inferior and anterior-posterior. Each lead represents a particular orientation in space.

The intrinsic structure of a wearable ECG patch prevents it from directly duplicating the standard 12-lead electrocardiogram which requires the electrode pairs be much further apart. However, one could use the locations of electrodes on the standard 12-lead ECG as a reference but would not be limited to it in using the multi-lead system of the present invention.

The wire free multi-lead wearable ECG devices of the present invention have significant advantages over the single lead device. A single lead wearable ECG patch may be acceptable when simply monitoring the cardiac rhythm but detrimental to the reliability of the ECG in other situations. Multi-lead affords us the opportunity to view the heart from a number of perspectives. Without the precordial leads, it would be impossible to detect abnormalities such as anterior or posterior myocardial infarction and differentiate left and right bundle branch block. Also, lead V1 often allows for best visualization of P waves. Additionally, the limb leads enable one to determine the cardiac axis and allow one to determine the presence of inferior myocardial infraction. It is indeed desirable to use a multi-lead in the case of infants, patients with dextrocardia or in the detection of right ventricular and posterior myocardial infraction.

The analyzed results from each device of the present invention can be displayed in different ways. The followings are two exemplary display methods:
(1) A summarized report: When the data is downloaded from the device to the computer or external system, the application software will extract all the analysis results and put them into a summarized report as well as collecting the device or patient's information. The analysis results include statistical analysis and classification on all the detected beats and the arrhythmias, and other events. All the detections are associated with real time for traceability.
(2) Graphic Display: Since all the original data are preserved, they can be displayed and printed in a conventional ECG graph for a physician to further analyze and diagnosis. The application software is configured to trace and display any data either by real time or by the classification.

The device, method and system of the present invention may be configured in various other embodiments due to its intelligent, reusable, wire-free and comfortable design as discussed above and further below.

The shape, color and size of the device 10 could be varied depending on the design according to other embodiments of the present invention. The components of the device could be located in a different area on the device substrate. For example, all the circuitry could be located in the center of the device instead of being distributed around the electrodes. In embodiments, the device is designed in such a way that the recording circuitry is located around one side of the electrode and battery with related circuitry residing around another electrode. The patient's information such as name and record number can be written on the bottom of the device or stored into the device electronically. Other technologies such as bar code or RFID could be used to store information about the device and patient. With the USB driver installed at a host computer, such a device is very similar to a USB memory stick. The ECG data, analyzed results, patient and device information can be retrieved by the system after the monitoring.

In other embodiments, the battery 11 could be a miniature rechargeable battery such as a lithium polymer battery and the battery recharging circuitry could also be built in around the battery. In a further embodiment, the USB interface could be used as power input connector to charge the built-in rechargeable battery if the device is designed for repeatable usage.

Overall, the device of the present invention is capable of monitoring and analyzing the ECG data in real-time while recording. In comparison to an event monitor, the device provides continuous recording of events through a real time embedded algorithm instead of being activated by physically pressing a button or switch. Such conventional event monitors which do record an event by manually activating a button are simply unreliable. It is difficult, inconvenient or too late for patients to push a switch when they feel the symptoms. Instead of pushing the button, a patient can have the time of a symptom recorded and tell the physician later to review the results using the device of the present invention. Whether or not the patient was aware of the symptom occurring, the device will record all the ECG data and the algorithm will automatically analyze the data.

The unique electrode interface design of the device according to an embodiment, makes it adaptable to different types of electrodes for various body size, skin sensitivity and monitoring purpose. It also makes the invented device capable of using multiple times with new electrodes as long as memory and battery are not run out. The device according to an embodiment of the present invention is intended for continuous recording and analysis from 24 hours upwards to 7 days, or even longer, depending on the built in memory size and life of the battery.

In an embodiment, the device is provided with an USB interface that is mainly for data uploading purpose, although the interface also can be used as a functional expansion port for many applications. By plugging in a small low power wireless module, the device can become part of a real time monitoring system. The USB interface can also be used to extend memory or charge the battery if a rechargeable battery is installed. The concept of using the USB port as a functional expansion interface makes the device potentially have more desirable functions in some particular applications. For instance, an ECG device can be connected to a cell phone by plugging in a miniature blue-tooth device. In emergency cases, a text message can be automatically sent out from a cell phone to predefined recipients with the help of the application software.

Furthermore, in an embodiment, the ECG device can be used as a storage media and physically kept within the patient's record in some applications. This can reduce the amount of database or data storage capability requirements for a hospital or data processing center. The recorded data is kept in an embedded non-volatile memory, which lends to long term persistent storage. Previous disclosed ECG devices are not reused in such a way, but are instead disposed.

The traditional portable ECG device either continuously records ECG data or selectively records data according to predefined conditions. Such ECG data analysis is performed at the base unit, remote station or data center with an ECG analyzer. It is always a major burden for the physician or data processing center to handle the long term unfiltered continuous ECG data because of the data size. Therefore, due to the embedded microcontroller, the device according to an embodiment of the present invention not only continuously records all the data but performs sophisticated ECG analysis during the recording on the ECG data. By embedding the real time ECG analysis algorithm in the device, it is capable of not only flagging the predefined threshold events but performing sophisticated ECG analysis.

As further detailed, the device according to an embodiment of the present invention contains a QRS detection algorithm. Since the QRS appears in most normal and abnormal ECG signals, it becomes the most important waveform to detect and characterize. The algorithm detects and measures the QRS duration, R-peak magnitude, RR interval, QT interval, onsets and offsets of the P wave and T wave. The reliable and unique recognition algorithm identifies the QRS by its intrinsic properties regardless of varied morphology or rhythm patterns. Many cardiac diseases can be identified based on the QRS recognition. For example, tachycardia heart conditions can be detected based on the wide and narrow QRS complex. One of the characteristics of atrial fibrillation (AF) episodes is determined by the absence of P waves before QRS-T complex.

The device according to an embodiment of the present invention also contains an ST-segment detection algorithm. The ST-segment level, ST slope and ST area are derived and calculated by the algorithm. The ST-segment measurement and analysis may indicate that there is deficiency in the blood supply to the heart muscle or a concern with myocardial abnormality.

The device of the present invention also performs arrhythmia analysis based on the results of QRS detection and ST-segment measurements. The ECG signals are classified according to its intrinsic parameters such as QRS duration and RR interval. For example, a premature ventricular contraction is characterized by a short RR interval coupled with a long QRS duration. The arrhythmias like asystole, paroxysmal bradycardia, premature ventricular contraction (PVC), atrial fibrillation (AF), ventricular tachycardia (VT) and ventricular fibrillation (VF) can be classified and diagnosed in the device.

The device itself of the present invention does not use any wireless technology to transmit and receive data from remote devices or gateways. The device does not necessarily require any external apparatus to configure it either. The device of the present invention is a self-contained, wearable, smart and miniaturized single lead ECG recorder and analyzer. It continuously detects, acquires, analyzes and records the ECG data into the integrated memory, as well as registers the recorded data with the analyzed results and embedded real time clock. There is no loss of raw data which allows the data to always be retrieved for further analysis. Multiple such devices compose of a multi-lead ECG system according to an embodiment of the present invention.

Throughout the description and drawings, example embodiments are given with reference to specific configurations. It will be appreciated by those of ordinary skill in the art that the present invention can be embodied in other specific forms. Those of ordinary skill in the art would be able to practice such other embodiments without undue experimentation. The scope of the present invention, for the purpose of the present patent document, is not limited merely to the specific example embodiments of the foregoing description.

We claim:

1. A self contained, wire-free, ambulatory ECG device for non-invasive continuous recording and analysis of ECG signals, the device comprising:
a device body having a flexible substrate, the substrate having embedded electronic components including a microcontroller, a memory embedded with a diagnosis software, a second memory for storing indexed ECG data, a real time clock, a functional switch, electronic circuitry, a plurality of conductors, a battery, and an interface for data communication;
two or more snap connectors integrated onto the substrate for engaging with two or more ECG electrodes separate from the device, the electrodes having a means to adhere to the skin of the body;
wherein the device body is detachable from the electrodes via the snap connectors and reusable with two or more new electrodes for a subsequent recording and analysis.

2. The device according to claim 1, wherein the flexible substrate is a printed circuit board on which the electronic components reside.

3. The device according to claim 1, wherein the flexible substrate is a soft polymeric material encapsulating all the embedded electronic components.

4. The device according to claim 1, wherein the interface for data communication is an embedded USB interface connector.

5. The device according to claim 1, further comprising a feedback indicator located on the device body for identifying a status of the device.

6. The device according to claim 1, wherein the embedded electronic components are positioned in a middle section of the substrate, one snap connector is positioned on one side of the embedded electronic components, and one or more snap connectors are positioned on an opposing side of the embedded electronic components.

7. The device according to claim 1, wherein the flexible substrate of the device body has two defined sections with a snap connector in each section, and wherein the embedded electronic components are distributed throughout the two sections.

8. The device according to claim 1, wherein the memory comprises an internal flash memory embedded with a cardiac diagnosis algorithm which detects, analyzes, and indexes the recorded ECG data in real time using the microcontroller.

9. The device according to claim 8, wherein the real time clock registers a first continuous ECG data at each time the device is activated for recording from a non recording mode, and indexes recorded ECG data with a time stamp.

10. The device according to claim 1, wherein the second memory comprises an external flash memory and the indexed ECG data comprises recorded raw ECG data, analyzed ECG data and real time clock information.

11. An ECG monitoring and recording system comprising:
a self-contained, wire free, ambulatory single lead ECG device which continuously records and analyzes ECG data, the device comprising:
electronic components operatively coupled and embedded within a flexible substrate of the device, the components including an embedded microcontroller, circuitry and conductors, a real time clock, a data communication interface, memory and software, and at least two electrode snap connectors disposed on the substrate of the device, and wherein the device indexes the ECG data and records the indexed ECG data;
a pair of electrodes for engaging with the snap connectors of the device, each of the pair of electrodes having a means for adhering to a patient's skin;
a computer host system having one or more processors for executing commands that direct operations of the computer system and software executing within the one or more processors that directs the one or more processors to:
obtain recorded indexed ECG data from the device through the data communications interface, the indexed ECG data comprising raw ECG data, analyzed ECG data and real time information;
extract the analyzed ECG data and the real time information from the indexed ECG data; and
display the analyzed ECG data from the device.

12. A multi-lead ECG monitoring and recording system comprising:
a plurality of self-contained, wire free, ambulatory single lead ECG devices, each device continuously records and analyzes ECG data, each device comprising electronic components operatively coupled, including an embedded microcontroller, circuitry and conductors, a real time clock, a functional switch, a battery, a data communication interface, memory and software, embedded within a flexible substrate of the device, each device having at least two electrode snap connectors disposed on the substrate of the device, and wherein each device indexes its ECG data and records its indexed ECG data;
a pair of electrodes for engaging with the snap connectors of the device, each of the pair of electrodes are adhered to a patient's skin;
a computer host system having one or more processors for executing commands that direct operations of the computer system and software executing within the one or more processors that directs the one or more processors to:
obtain recorded indexed ECG data from each device through the data communications interface, the indexed ECG data comprising raw ECG data, analyzed ECG data and real time information;
extract the analyzed ECG data and real time information from the indexed data of each device;
calculate a relative time offset amongst each real time clock of each device;
update all real time analyzed ECG data of the devices with the calculated time offset;
correlate the analyzed ECG data from each device into a multi-lead ECG data array;
combine the analyzed ECG data of each of the devices and
display the analyzed ECG data from the devices.

13. The system according to claim 12, wherein the one or more processors of the computer host system is directed to further generate summary reports and statistical graphs based on the analyzed ECG data of the devices.

14. The system according to claim 12, wherein a synchronization of each of the plurality of single lead ECG devices is conducted prior to calculating a relative time offset, the synchronization comprising:
after each device is connected with a set of connection wires, each device is set into a synchronization state via the functional switch;
a designated master device sends out a triggering pulse to all devices;
each device receives the triggering pulse and records a time from its embedded real time clock corresponding to the triggering pulse received;
upon successful synchronization, every device is returned to an active state via the functional switch and the connecting wires are removed.

15. The system according to claim 12, wherein a synchronization of each of the plurality of single lead ECG devices is conducted prior to calculating a relative time offset, the synchronization comprising:
designating a reference time from the time clock of the computer host system;
registering each real time clock of the devices to correspond with the same reference time of the computer host system through the data communications interface; and
recording the relative time offset between the real time clock of each device and the same reference time of the computer host system.

16. The system according to claim 12, wherein the data communication interface is a USB interface through which the host computer retrieves the ECG data from each device through a USB port on the host computer.

17. The system according to claim 12, wherein the memory comprises an internal flash memory and an external flash memory,
the internal flash memory embedded with a cardiac diagnosis algorithm which detects, analyzes, and indexes the recorded ECG data in real time using the microcontroller; and
the external flash memory stores the indexed ECG data.

18. The system according to claim 12, wherein the functional switch operates to control activation of continuous ECG recording and a separate synchronization process.

19. A computer implemented method for continuous ambulatory ECG recording and analysis, using a wire free, self contained ambulatory ECG device having embedded electronic components operatively coupled, including a microcontroller, memory with an embedded diagnosis algorithm, a real time clock, a functional switch, a battery, electronic circuitry, a plurality of conductors, and an interface for data communication, the method comprising the steps of:
adhering a pair of electrodes onto a patient's skin;
engaging the device with the pair of electrodes through a set of electrode snap connectors integrated onto a bottom side of a flexible substrate of the device;
acquiring ECG signals through the electrodes and recording the signals as raw ECG data temporarily in a memory;
electronically analyzing the raw ECG data in real-time resulting in analyzed ECG data, through the microcontroller;
electronically registering the analyzed ECG data with a time stamp with the embedded real time clock, through the microcontroller;
electronically indexing the raw ECG data with the analyzed ECG data and the real time clock, through the microcontroller; and
electronically storing the indexed and analyzed ECG data into the memory.

20. The method according to claim 19, further comprising:
electronically uploading the analyzed ECG data from the memory into an external host system through the data communications interface on the device; and
electronically displaying the analyzed ECG data through the host system.

21. The method according to claim 19, wherein the steps of acquiring, analyzing, registering, indexing and storing continuously for up to 7 seven days.

22. The method according to claim 19, wherein a plurality of the same ECG devices are arranged with a pair of electrodes for each device for multi-lead monitoring and the method further comprises synchronizing each device.
